(19) United States
(12) Patent Application Publication
(10) Pub. No.: US 2003/0099938 A1
(43) Pub. Date: May 29, 2003

(54) NOVEL HOMEODOMAIN PROTEIN

(76) Inventors: DAVID J. ANDERSON, ALTADENA, CA (US); TETSUICHIRO SAITO, TSUKUBA (JP)

Correspondence Address: FLEHR HOHBACH TEST ALBRITTON & HERBERT, FOUR EMBARCADERO CENTER, SUITE 3400, SAN FRANCISCO, CA, 94111

(21) Appl. No.: 08/701,278

(22) Filed: Aug. 22, 1996

Related U.S. Application Data
(60) Provisional application No. 60/023,280, filed on Jul. 25, 1996.

(51) Int. Cl. C12Q 1/68; G01N 33/53; C07H 21/04; C12P 21/02; C12N 5/06; C07K 14/705

(52) U.S. Cl. 435/6; 536/23.5; 435/69.1; 435/320.1; 435/325; 530/350; 435/7.1

(57) ABSTRACT

The invention relates to novel paired homeodomain proteins, nucleic acids and antibodies, and to a novel method of differential reverse-transcriptase based polymerase chain reaction (dRT-PCR).

### FIELD OF THE INVENTION

The invention relates to novel paired homeodomain proteins, nucleic acids and antibodies, and to a novel method of differential reverse-transcriptase based polymerase chain reaction (dRT-PCR).

### BACKGROUND OF THE INVENTION

The study of cellular diversification during neurogenesis requires markers to identify different neural cell types. Recently it has become clear that transcription factors can serve as useful markers of neuronal identity. For example, the bHLH protein MASH1 identifies autonomic progenitors in the PNS (Lo et al., 1994). Similarly Islet-1 (Ericson et al., 1992) and additional recently-characterized proteins in the lim homeodomain family mark subsets of functionally-distinct motor neurons (Tsuchida et al., 1994). These data suggests that the diversity of neuronal cell types can be defined in terms of an underlying diversity of expression of members of a transcription factor gene family.

Polymerase chain reaction (PCR)-based cloning using degenerate oligonucleotide primers has proven useful as a method to rapidly isolate members of a gene family (see, for example, (Libert et al., 1989; Wilkie and Simon, 199 1)). When cDNA rather than genomic DNA is used as the template, the method can selectively identify those members of a gene family expressed in a given tissue or cell type (Johnson et al., 1990; Lai and Lemke, 1991). A limitation of this approach is that one must often sequence tens if not hundreds of PCR products to identify novel genes, and then examine their expression patterns to identify those appropriate for further study. This makes it labor-intensive to apply this approach to several gene families simultaneously.

Neuropeptides such as CGRP (Murphy et al., 1991) and Substance P (Ito et al., 1993) have been used as sensory markers, but these neuropeptides are not in fact sensory neuron-specific: for example, they can be induced in cultured sympathetic neurons by some cytokines (Fann and Patterson, 1994). In the absence of definitive sensory neuron markers, unambiguous identification of sensory neurons in mammalian neural crest cultures has been difficult (Matsumoto, 1994).

Accordingly, it is an object of the present invention to provide a marker to identify neurons in the peripheral sensory lineage. Accordingly, the invention provides recombinant DRG11 proteins and variants thereof, and to produce useful quantities of these DRG11 proteins using recombinant DNA techniques.

It is a further object of the invention to provide recombinant nucleic acids encoding DRG11 proteins, and expression vectors and host cells containing the nucleic acid encoding the DRG11 protein.

An additional object of the invention is to provide polyclonal and monoclonal antibodies directed against DRG11 proteins.

A further object of the invention is to provide methods for producing the DRG11 proteins.

An additional object is to provide novel methods for the identification of gene family members which are specifically expressed in a given tissue or cell type.

### SUMMARY OF THE INVENTION

In accordance with the objects outlined above, the present invention provides recombinant nucleic acids encoding a DRG11 protein, expression vectors containing the nucleic acids and host cells transformed with the expression vectors.

The invention further provides methods of producing a DRG11 protein comprising culturing a host cell transformed with an expressing vector comprising a DGR11 nucleic acid and expressing the nucleic acid to produce a DRG11 protein.

The invention additionally provides DRG11 proteins and antibodies.

The invention also provides methods for determining the differential expression of a gene in different cell types or tissues. First, libraries of nucleic acids from a plurality of different cell types or tissues are synthesized using a set of primers. A portion of the library obtained from a first of said different cell types or tissue is subcloned to form a subclone library. Then, members of the subclone library are separately contacted with probes, each of which comprise one of the libraries. The nucleic acids in the libraries are labeled and the contacting is under conditions which permit the hybridization of the labeled nucleic acids to complementary nucleic acids, if present, in the subclone library. Next, a determination is made whether hybridization has occurred for each of the probes for members of the subclone library as an indication of the differential expression of a gene expressed by said first cell type or tissue.

### BRIEF DESCRIPTION OF THE DRAWINGS

FIGS. 1A and 1B depict the differential reverse-transcriptase polymerase chain reaction (dRT-PCR) used herein. FIG. 1A is a schematic representation of the dRT-PCR procedure. FIG. 1B is an example of differential hybridization.

FIG. 2 depicts the nucleotide sequence of rat DRG11.

FIG. 3 depicts the amino acid sequence of rat DRG11.

FIG. 4 depicts a homology lineup of the homeodomain sequences among paired homeodomain proteins. The sources of the sequences illustrated are: Phox2 (Valarche et al., 1993); Cart1 (Zhao et al., 1993); Phox1 (Grueneberg et al., 1992); S8 (Opstelten et al., 1991); Pax6 (Walther and Gruss, 1991); Da1 (Schneitz et al., 1993); Dprd (Frigerio et al., 1986); Drepo (Xiong et al., 1994); and Cunc4 (Miller et al., 1992).

### DETAILED DESCRIPTION OF THE INVENTION

The present invention provides novel DRG11 proteins. In a preferred embodiment, the DRG11 proteins are from vertebrates, more preferably from mammals, and in the preferred embodiment, from rats or humans. However, using the techniques outlined below, DRG11 proteins from other organisms may also be obtained.

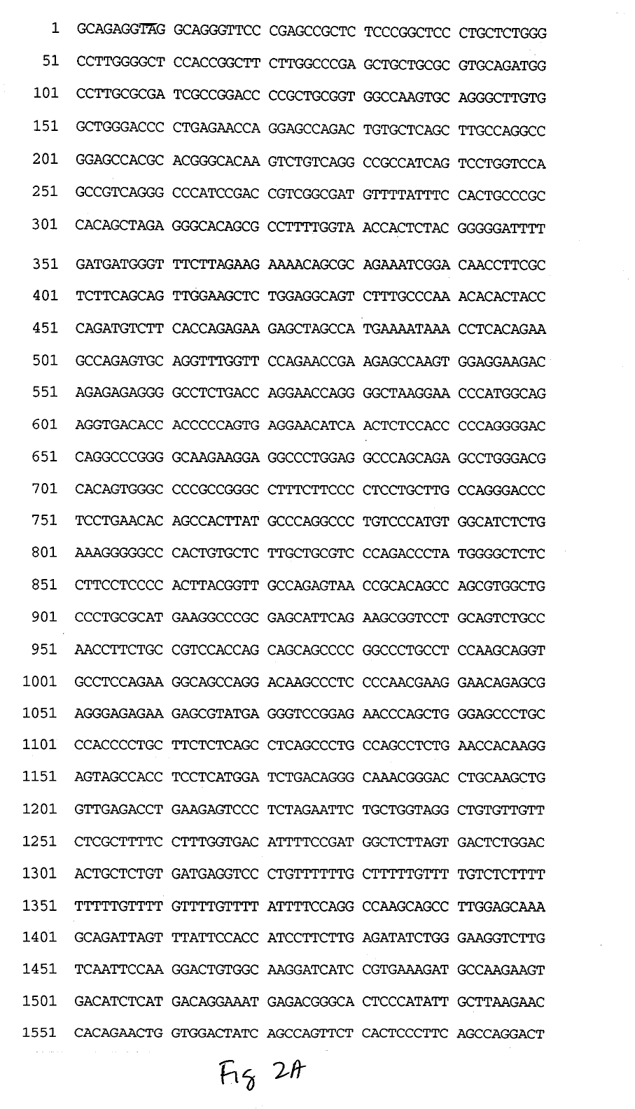

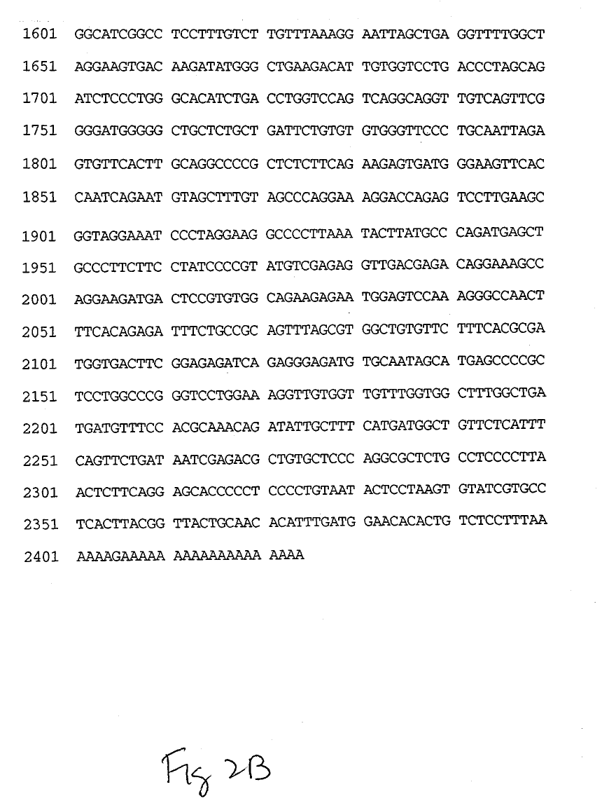

A DRG11 protein of the present invention may be identified in several ways. A DRG11 nucleic acid or DRG11 protein is initially identified by substantial nucleic acid and/or amino acid sequence homology to the sequences shown in FIGS. 2 or 3. Such homology can be based upon the overall nucleic acid or amino acid sequence.

As used herein, a protein is a “DRG11 protein” if the overall homology of the protein sequence to the amino acid sequence shown in FIG. 3 is preferably greater than about 70%, more preferably greater than about 80% and most preferably greater than 90%. In some embodiments the homology will be as high as about 95 or 98%. This homology will be determined using standard techniques known in the art, such as the Best Fit sequence program described by Devereux et al., Nucl. Acid Res. 12:387-395 (1984) or the BLASTX program (Altschul et al., J. Mol. Biol. 215, 403-410). The alignment may include the introduction of gaps in the sequences to be aligned. In addition, for sequences which contain either more or fewer amino acids than the protein shown in the Figures, it is understood that the percentage of homology will be determined based on the number of homologous amino acids in relation to the total number of amino acids. Thus, for example, homology of sequences shorter than that shown in the Figures, as discussed below, will be determined using the number of amino acids in the shorter sequence.

DRG11 proteins may be identified in one aspect by significant homology to the areas other than the homeodomain, i.e. the N- and C-terminal portions of the sequences depicted in the Figures. This homology is preferably greater than about 70%, with greater than about 80% being particularly preferred and greater than about 90% being especially preferred. In some cases the homology will be greater than about 90 to 95 or 98%.

In addition, a DRG11 protein preferably also has significant homology to the DRG11 homeodomain as described herein. This homology is preferably greater than about 70%, with greater than about 80% being particularly preferred and greater than about 90% being especially preferred. In some cases the homology will be greater than about 90 to 95 or 98%.

DRG11 proteins of the present invention may be shorter or longer than the amino acid sequences shown in the Figures. Thus, in a preferred embodiment, included within the definition of DRG11 proteins are portions or fragments of the sequences shown in FIG. 3.

DRG11 proteins may also be identified as being encoded by DRG11 nucleic acids. Thus, DRG11 proteins are encoded by nucleic acids that will hybridize to the sequence depicted in FIG. 2, as outlined herein.

In a preferred embodiment, when the DRG11 protein is to be used to generate antibodies, the DRG11 protein must share at least one epitope or determinant with the full length protein shown in FIG. 3. By “epitope” or “determinant” herein is meant a portion of a protein which will generate and/or bind an antibody. Thus, in most instances, antibodies made to a smaller DRG11 protein will be able to bind to the full length protein. In a preferred embodiment, the epitope is unique; that is, antibodies generated to a unique epitope show little or no cross-reactivity; thus, for example, the antibodies preferably do not bind the homeodomain. In a preferred embodiment, the antibodies are generated to the N- and C-terminal portion of the DRG11 molecule. The DRG11 antibodies of the invention specifically bind to DRG11 proteins. By “specifically bind” herein is meant that the antibodies bind to the protein with a binding constant in the range of at least 10⁴-10⁶ M⁻¹, with a preferred range being 10⁷-10⁹ M⁻¹.

In the case of the nucleic acid, the overall homology of the nucleic acid sequence is commensurate with amino acid homology but takes into account the degeneracy in the genetic code and codon bias of different organisms. Accordingly, the nucleic acid sequence homology may be either lower or higher than that of the protein sequence. Thus the homology of the nucleic acid sequence as compared to the nucleic acid sequence of FIG. 2 is preferably greater than 60%, more preferably greater than about 65%, particularly greater than about 70% and most preferably greater than 80%. In some embodiments the homology will be as high as about 90 to 95 or 98%.

In a preferred embodiment, a DRG11 nucleic acid encodes a DRG11 protein. As will be appreciated by those in the art, due to the degeneracy of the genetic code, an extremely large number of nucleic acids may be made, all of which encode the DRG11 proteins of the present invention. Thus, having identified a particular amino acid sequence, those skilled in the art could make any number of different nucleic acids, by simply modifying the sequence of one or more codons in a way which does not change the amino acid sequence of the DRG11.

In one embodiment, the nucleic acid homology is determined through hybridization studies. Thus, for example, nucleic acids which hybridize under high stringency to the nucleic acid sequence shown in FIG. 2 or their complements are considered DRG11 genes. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference.

In another embodiment, less stringent hybridization conditions are used; for example, moderate or low stringency conditions may be used, as are known in the art; see Maniatis and Ausubel, supra.

The DRG11 proteins and nucleic acids of the present invention are preferably recombinant. As used herein, “nucleic acid” may refer to either DNA or RNA, or molecules which contain both deoxy- and ribonucleotides. The nucleic acids include genomic DNA, cDNA and oligonucleotides including sense and antisense nucleic acids. Such nucleic acids may also contain modifications in the ribose-phosphate backbone to increase stability and half life of such molecules in physiological environments.

The nucleic acid may be double stranded, single stranded, or contain portions of both double stranded or single stranded sequence. By the term “recombinant nucleic acid” herein is meant nucleic acid, originally formed in vitro, in general, by the manipulation of nucleic acid by endonucleases, in a form not normally found in nature. Thus an isolated DRG11 nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Similarly, a “recombinant protein” is a protein made using recombinant techniques, i.e. through the expression of a recombinant nucleic acid as depicted above. A recombinant protein is distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises at least about 75% by weight of the total protein, with at least about 80% being preferred, and at least about 90% being particularly preferred. The definition includes the production of a DRG11 protein from one organism in a different organism or host cell. Alternatively, the protein may be made at a significantly higher concentration than is normally seen, through the use of a inducible promoter or high expression promoter, such that the protein is made at increased concentration levels. Alternatively, the protein may be in a form not normally found in nature, as in the addition of an epitope tag or amino acid substitutions, insertions and deletions, as discussed below.

Also included with the definition of DRG11 protein are other DRG11 proteins of the DRG11 family, and DRG11 proteins from other organisms, which are cloned and expressed as outlined below. Thus, probe or degenerate polymerase chain reaction (PCR) primer sequences may be used to find other related DRG11 proteins from humans or other organisms. As will be appreciated by those in the art, particularly useful probe and/or PCR primer sequences include the unique areas of the DRG11 nucleic acid sequence. Thus, useful probe or primer sequences may be designed to the N- and C-terminal portions of the sequence. As is generally known in the art, preferred PCR primers are from about 15 to about 35 nucleotides in length, with from about 20 to about 30 being preferred, and may contain inosine as needed. The conditions for the PCR reaction are well known in the art.

Once the DRG11 nucleic acid is identified, it can be cloned and, if necessary, its constituent parts recombined to form the entire DRG11 protein nucleic acid. Once isolated from its natural source, e.g., contained within a plasmid or other vector or excised therefrom as a linear nucleic acid segment, the recombinant DRG11 nucleic acid can be further used as a probe to identify and isolate other DRG11 nucleic acids. It can also be used as a “precursor” nucleic acid to make modified or variant DRG11 nucleic acids and proteins.

Using the nucleic acids of the present invention which encode a DRG11 protein, a variety of expression vectors are made. The expression vectors may be either self-replicating extrachromosomal vectors or vectors which integrate into a host genome. Generally, these expression vectors include transcriptional and translational regulatory nucleic acid operably linked to the nucleic acid encoding the DRG11 protein. “Operably linked” in this context means that the transcriptional and translational regulatory DNA is positioned relative to the coding sequence of the DRG11 protein in such a manner that transcription is initiated. Generally, this will mean that the promoter and transcriptional initiation or start sequences are positioned 5′ to the DRG11 protein coding region. The transcriptional and translational regulatory nucleic acid will generally be appropriate to the host cell used to express the DRG11 protein; for example, transcriptional and translational regulatory nucleic acid sequences from Bacillus are preferably used to express the DRG11 protein in Bacillus. Numerous types of appropriate expression vectors, and suitable regulatory sequences are known in the art for a variety of host cells.

In general, the transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, the expression vector may comprise additional elements. For example, the expression vector may have two replication systems, thus allowing it to be maintained in two organisms, for example in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

In addition, in a preferred embodiment, the expression vector contains a selectable marker gene to allow the selection of transformed host cells. Selection genes are well known in the art and will vary with the host cell used.

The DRG11 proteins of the present invention are produced by culturing a host cell transformed with an expression vector containing nucleic acid encoding a DRG11 protein, under the appropriate conditions to induce or cause expression of the DRG11 protein. The conditions appropriate for DRG11 protein expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

Appropriate host cells include yeast, bacteria, archebacteria, fungi, and insect and animal cells, including mammalian cells. Of particular interest are Drosophila melangaster cells, Saccharomyces cerevisiae and other yeasts, E. coli, Bacillus subtilis, SF9 cells, Cl 29 cells, 293 cells, Neurospora, BHK, CHO, COS, and HeLa cells, DAVID, WHAT ARE PREFERRED HOST CELLS?

In a preferred embodiment, the DRG11 proteins are expressed in mammalian cells. Mammalian expression systems are also known in the art. A mammalian promoter is any DNA sequence capable of binding mammalian RNA polymerase and initiating the downstream (3′) transcription of a coding sequence for DRG11 protein into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenlytion signals include those derived form SV40.

The methods of introducing exogenous nucleic acid into mammalian hosts, as well as other hosts, is well known in the art, and will vary with the host cell used. Techniques include the use of viruses such as retroviruses and adenoviruses, dextran-mediated transfection, calcium phosphate precipitation, polybrene mediated transfection, protoplast fusion, electroporation, encapsulation of the polynucleotide(s) in liposomes, and direct microinjection of the DNA into nuclei.

In a preferred embodiment, DRG11 proteins are expressed in bacterial systems. Bacterial expression systems are well known in the art.

A suitable bacterial promoter is any nucleic acid sequence capable of binding bacterial RNA polymerase and initiating the downstream (3′) transcription of the coding sequence of DRG11 protein into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tac promoter is a hybrid of the trp and lac promoter sequences. Furthermore, a bacterial promoter can include naturally occurring promoters of non-bacterial origin that have the ability to bind bacterial RNA polymerase and initiate transcription.

In addition to a functioning promoter sequence, an efficient ribosome binding site is desirable. In E. coli, the ribosome binding site is called the Shine-Delgarno (SD) sequence and includes an initiation codon and a sequence 3-9 nucleotides in length located 3-11 nucleotides upstream of the initiation codon.

The expression vector may also include a signal peptide sequence that provides for secretion of the DRG11 protein in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria).

The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways.

These components are assembled into expression vectors. Expression vectors for bacteria are well known in the art, and include vectors for Bacillus subtilis, E coli, Streptococcus cremoris, and Streptococcus lividans, among others.

The bacterial expression vectors are transformed into bacterial host cells using techniques well known in the art, such as calcium chloride treatment, electroporation, and others.

In one embodiment, DRG11 proteins are produced in insect cells. Expression vectors for the transformation of insect cells, and in particular, baculovirus-based expression vectors, are well known in the art.

In a preferred embodiment, DRG11 protein is produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica. Preferred promoter sequences for expression in yeast include the inducible GAL1,10 promoter, the promoters from alcohol dehydrogenase, enolase, glucokinase, glucose-6-phosphate isomerase, glyceraldehyde-3-phosphate-dehydrogenase, hexokinase, phosphofructokinase, 3-phosphoglycerate mutase, pyruvate kinase, and the acid phosphatase gene. Yeast selectable markers include ADE2, HIS4, LEU2, TRP1, and ALG7, which confers resistance to tunicamycin; the neomycin phosphotransferase gene, which confers resistance to G418; and the CUP1 gene, which allows yeast to grow in the presence of copper ions.

The DRG11 protein may also be made as a fusion protein, using techniques well known in the art. Thus, for example, for the creation of monoclonal antibodies, if the desired epitope is small, the DRG11 protein may be fused to a carrier protein to form an immunogen. Alternatively, the DRG11 protein may be made as a fusion protein to increase expression, or for other reasons.

Also included within the definition of DRG11 proteins of the present invention are amino acid sequence variants. These variants fall into one or more of three classes: substitutional, insertional or deletional variants. These variants ordinarily are prepared by site specific mutagenesis of nucleotides in the DNA encoding the DRG11 protein, using cassette or PCR mutagenesis or other techniques well known in the art, to produce DNA encoding the variant, and thereafter expressing the DNA in recombinant cell culture as outlined above. However, variant DRG11 protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the DRG11 protein amino acid sequence. The variants typically exhibit the same qualitative biological activity as the naturally occurring analogue, although variants can also be selected which have modified characteristics as will be more fully outlined below.

While the site or region for introducing an amino acid sequence variation is predetermined, the mutation per se need not be predetermined. For example, in order to optimize the performance of a mutation at a given site, random mutagenesis may be conducted at the target codon or region and the expressed DRG11 variants screened for the optimal combination of desired activity. Techniques for making substitution mutations at predetermined sites in DNA having a known sequence are well known, for example, M13 primer mutagenesis and PCR mutagenesis. Screening of the mutants is done using assays of DRG11 protein activities.

Amino acid substitutions are typically of single residues; insertions usually will be on the order of from about 1 to 20 amino acids, although considerably larger insertions may be tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. However, larger changes may be tolerated in certain circumstances. When small alterations in the characteristics of the DRG11 protein are desired, substitutions are generally made in accordance with the following chart:

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

The variants typically exhibit the same qualitative biological activity and will elicit the same immune response as the naturally-occurring analogue, although variants also are selected to modify the characteristics of the DRG11 proteins as needed. Alternatively, the variant may be designed such that the biological activity of the DRG11 protein is altered.

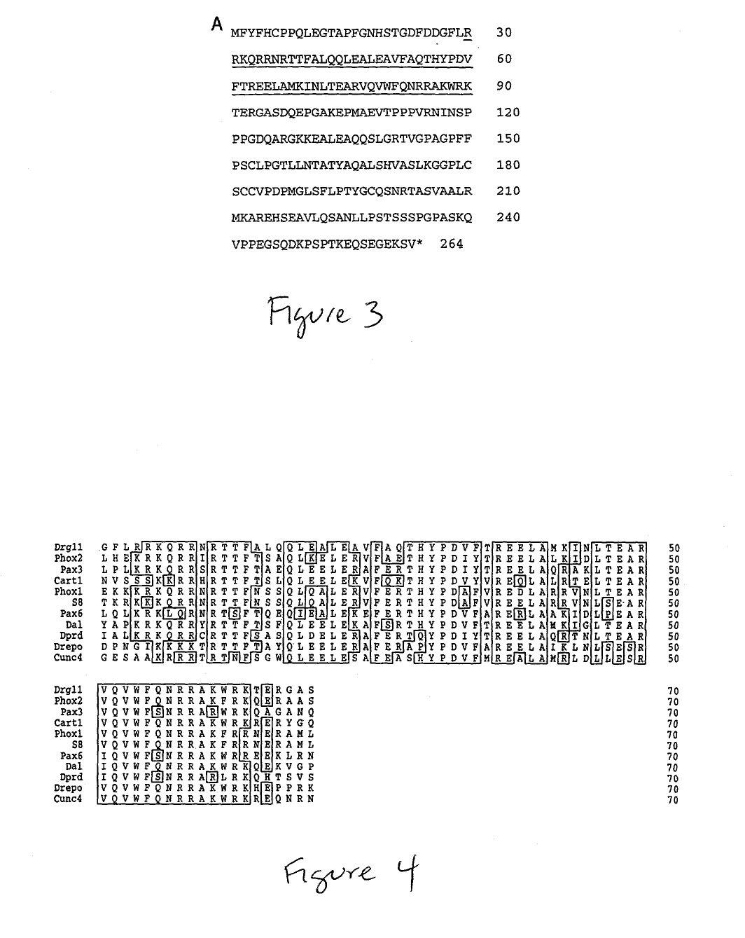

In one embodiment, homeodomain variants are made. In one embodiment, a homeodomain may be eliminated entirely. Alternatively, any or all of the amino acids of a homeodomain may be be altered or deleted. In a preferred embodiment, one or more of the amino acids of the homeodomain are substituted by other amino acids. Thus, amino acids corresponding to the rat DRG11 homeodomain residues, depicted in either FIG. 3 or 4 may be altered.

In addition, as outlined in the Examples, the homophilic DRG11 1 cellular adhesion is dependent on the presence of divalent cations; thus, the metal binding properties of the binding domain may be altered.

In one embodiment, the DRG11 nucleic acids, proteins and antibodies of the invention are labelled. By “labelled” herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position.

In a preferred embodiment, the DRG11 protein is purified or isolated after expression. DRG11 proteins may be isolated or purified in a variety of ways known to those skilled in the art depending on what other components are present in the sample. Standard purification methods include electrophoretic, molecular, immunological and chromatographic techniques, including ion exchange, hydrophobic, affinity, and reverse-phase HPLC chromatography, and chromatofocusing. For example, the DRG11 protein may be purified using a standard anti-DRG11 antibody column. Ultrafiltration and diafiltration techniques, in conjunction with protein concentration, are also useful. For general guidance in suitable purification techniques, see Scopes, R., Protein Purification, Springer-Verlag, NY (1982). The degree of purification necessary will vary depending on the use of the DRG11 protein. In some instances no purification will be necessary.

Once expressed and purified if necessary, the DRG11 proteins are useful in a number of applications.

In a preferred embodiment, the DRG11 gene, protein or antibody is used as a marker for detecting the presence or absence of sensory neurons. As outlined herein, DRG11 is specifically expressed in sensory neurons but not in autonomic neurons or glia. Unexpectedly, DRG11 is also expressed in a subset the known functional targets of sensory neurons in the spinal cord. Therefore, this putative transcription factor can be used as a molecular marker to identify neurons in the peripheral sensory lineage. All or part of the sequence can be used, although preferably unique sequences are used, as outlined herein.

Since the pattern of expression of DRG11 in the central nervous system suggests that DRG11 may function in regulating some aspect of the connectivity between these neurons and their central targets, DRG11 proteins, nucleic acids and antibodies are useful in the screening and diagnosis of the presence or absence of DRG11 in a variety of cell types.

The DRG11 protein is also useful in screens to identify antagonists and agonists of DRG11, as will be appreciated by those in the art.

In one embodiment, the DRG11 proteins of the present invention may be used to generate polyclonal and monoclonal antibodies to DRG11 proteins, which are useful as described below. Similarly, the DRG11 proteins can be coupled, using standard technology, to affinity chromatography columns. These columns may then be used to purify DRG11 antibodies. In a preferred embodiment, the antibodies are generated to epitopes unique to the DRG11 protein; that is, the antibodies show little or no cross-reactivity to other proteins. These antibodies find use in a number of applications. For example, the DRG11 antibodies may be coupled to standard affinity chromatography columns and used to purify DRG11 proteins.

The presence or absence or DRG11 may be assayed or detected using labelled DRG11 proteins, antibodies or nucleic acids. For example, methods are provided for detecting a DRG11 protein in a target sample comprising contacting a labelled polypeptide such as an antibody which will specifically bind to a DRG11 protein with the target sample and assaying for the presence of binding between the labelled polypeptide and DRG11, if present, in the target sample. The contacting is done under conditions which allow binding to DRG11.

The present invention also provides novel methods for differential reverse-transcriptase-based polymerase chain reaction (dRT-PCR). In a preferred embodiment, dRT-PCR allows the identification of members of a gene family which are specifically expressed in a given tissue or cell type. This method allows the determination of the differential expression of a gene in different cell types or tissues. The method first synthesizes libraries of nucleic acids from a plurality of different cell types or tissues using a set of primers. In a preferred embodiment, the synthesis is done using polymerase chain reaction (PCR), as is known in the art.

In a preferred embodiment, the primers are degenerate oligonucleotide primers flanking or within a conserved domain from a given gene family, although this is not required. Preferably, the primers are generated to regions within a conserved domain from a family or superfamily of genes known in the art. Preferred conserved domains include, but are not limited to, transcription factor or DNA binding gene families such as homeodomain regions, Ets-domain (Nye et al., 1992), forkhead (Lai et al., 1991), scalloped/TEF (Campbell et al., 1992), leucine zipper, and zinc finger domains.

A portion of the library obtained from at least one, i.e. the first, different cell types or tissue is then subcloned to form a subclone library. The first different cell type is generally the one in which information regarding differential expression is sought, i.e. that contains the gene(s) of interest.

Once subcloned, members of the subclone library are probed. The probes are labelled nucleic acids derived from the above described libraries from the first step. The conditions for probe hybridization (or contact) are chosen under conditions which permit the hybridization of the labeled nucleic acids to complementary nucleic acids, if present, in the subclone library.

The patterns of hybridization using the different probes on the subclone library allow the determination of the differential expression of different genes, as outlined in the Examples, thus serving as an indication of the differential expression of a gene expressed by the first cell type or tissue.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLE

### Identification of DRG11 by dRT-PCR

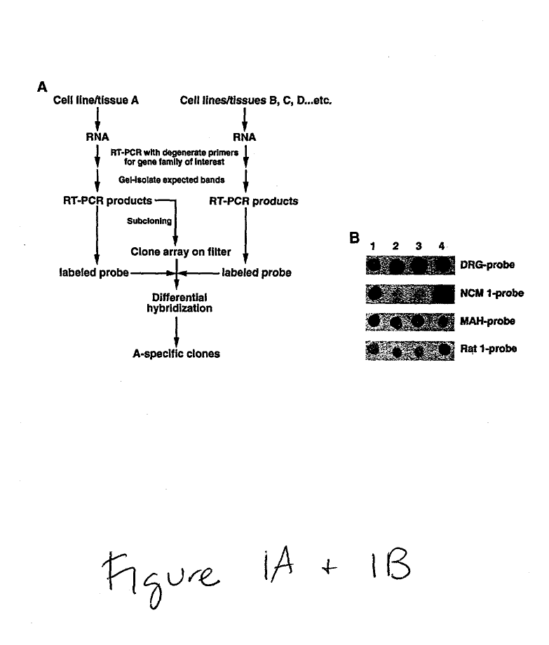

A schematic outline of the differential RT-PCR (dRT-PCR) procedure is illustrated in FIG. 1A. cDNA from two or more cell types or tissues is amplified using degenerate oligonucleotide primers flanking a conserved domain from a given gene family. The PCR products from the “positive” tissue of interest (“A-cell” in FIG. 1A) are subcloned, and the colonies are grown in a 12×8 array in a microtiter plate. Multiple replicas of this array are prepared by spotting small aliquots of each liquid bacterial culture onto nylon filters (Wilkie and Simon, 1991). These filters are then annealed with [³²P] probes made from the same RT-PCR products, as well as from the RT-PCR products derived from one or more “negative” tissues (“B, C, D . . . etc.-cells” in FIG. 1A). Clones displaying differential hybridization (A⁺B⁻C⁻D⁻) are picked for sequencing.

Molecular cloning was performed according to standard procedures (Sambrook et al., 1989), with minor modifications. Total RNA was prepared by acid guanidinum thiocyanate method as described (Chomczynski and Sacchi, 1987) with slight modification. cDNA was synthesized from 2 μg of total RNA in 50 μl by using random hexamer primers and reverse transcriptase, and 1 μl of this reaction mixture was used for PCR. Degenerate oligonucleotide primers corresponding to the sequence coding for amino acids FTAYQLE and the complementary sequence coding for amino acids QVWFQNR (N-terminal and C-terminal portions of the paired type homeodomain from the Drosophila protein RK2/repo (Campbell et al., 1994; Xiong et al., 1994)) were used for PCR: CGGGATCCTT(TC)ACIGCITA(TC)CA(GA)(TC)TIGA and CGGAATTC(GT)(GA)TT(TC)TG(GA)AACCAIAC(TC)TG. DNA was amplified by Taq DNA polymerase under the following conditions: 5 reduced stringency cycles using: 94° C. for 1 min, 42° C. for 1 min, 55° C. for 1 min, and 72° C. for 1 min; followed by 33 cycles using: 94° C. for 1 min, 55° C. for 1 min, and 72° C. for 1 min. PCR products were fractionated by agarose gels and DNA fragments of the expected size (150 bp) were purified using the Mermaid kit (Bio101). Purified DNA fragments were reamplified using 28 cycles of 94° C. for 1 min, 55° C. for 1 min, and 72° C. for 1 min, purified by phenol extraction, and digested by EcoRI and BamHI. Digested DNA fragments with a size of 150 bp were purified from agarose gels (see above). An aliquot of the digested DNA fragments was ligated into pBluescript for subcloning and transformed into E. coli. Another aliquot of these DNA fragments was saved for use as a probe in differential hybridization. Each transformant was transferred into 70 Al of LB-amp medium in a well of a 96-well plate and cultured at 37° C. for 14 hr with shaking. 5 μl of the liquid culture was spotted onto replica filters (Genescreen, Dupont) using a multichannel pipettor. The filter was treated according to the manufacturer's protocol and hybridized with a ³²P-labelled probe prepared by random-primed labelling of the other aliquot of the restriction-digested PCR products. Nucleotide sequences of the clones which showed differential distribution were determined.

Southern Blotting of PCR Products

PCR products of RT-PCR were fractionated by agarose gel electrophoresis, transferred onto replicate Genescreen filters and hybridized with ³²P-labelled probes derived from the DNA fragments encoding the homeodomains of DRG11, Pax3 or NCM3. As a control for the amount of amplified cDNA in the gel blot experiments, parallel aliquots were amplified for β-actin using gene-specific primers. The PCR product corresponded to a 310 bp fragment spanning nucleotides 767-1077 of the rat β-actin mRNA, and was amplified with the following primers: TCATGAAGTGTGACGTTGACATCC and GTAAAACGCAGCTCAGTAACAGTC. Conditions were 20 cycles of: 94° C. for 1 min., 60° C. for 1 min., 72° C. for 1 min.

For simplicity, examples of only four clones (FIG. 1B, columns 1-4) derived from paired homeodomain (PHD)-primer amplification of embryonic day 13.5 (E13.5) rat DRG cDNA, annealed in quadruplicate with four different probes, are illustrated. The probes used were PHD-amplified PCR products from: DRG, NCM-1 cells (a glial progenitor cell line (Lo et al., 1990)), MAH cells (a sympathoadrenal progenitor cell line (Birren and Anderson, 1990)) and Rat-1 fibroblasts (a non-neural cell line). Two pieces of information are derived from this screen. First, the relative intensities of the hybridization signals exhibited by a single clone annealed with multiple probes gives an indication of its relative abundance within its gene family in different cells or tissues (FIG. 1B, read vertically). Second, the relative intensities of the hybridization signals exhibited by multiple clones annealed with a single probe provides an indication of their relative abundance within that cell or tissue type (FIG. 1B, read horizontally). Taken together, such information provides a unique “fingerprint” for each clone. Such fingerprint data allows a preliminary assessment of the number of distinct differentially-expressed sequences within an array of clones.

In the example shown in FIG. 1B, clone 1 is strongly expressed in DRG, weakly in NCM-1 cells, and not above background in MAH and Rat-1 cells (background is defined by the weakest hybridization signal observed on a clone array with a given probe); this clone was later identified as Pax3 (Goulding et al., 1991). Clones 2 and 3 are expressed strongly in DRG, and not above background in NCM-1, MAH and Rat-1 cells; this clone was subsequently identified as a novel PHD protein which we have called DRG11. Clone 4 is expressed in DRG and NCM-1 cells, and weakly in Rat-1 cells.

To further confirm the specificity of expression of the clones identified in the initial differential screen, the hybridization procedure was reversed: inserts from three of the clones (DRG11, Pax3 and NCM3) were used as hybridization probes on gel blots of total PHD RT-PCR products from various cell lines, or tissues at different stages (data not shown). Consistent with the clone blot data (FIG. 1B), the DRG11 probe annealed to a single band in PHD RT-PCR products from DRG, but not in MAH, Rat1 or NCM-1 cells. By this analysis, DRG 11 mRNA appears to be expressed in sensory ganglia but not in cell lines representing sympathetic neuron or glial precursors, or in fibroblasts. As expected from the clone blot analysis, hybridization patterns distinct from that of DRG11 were obtained with the Pax3 and NCM3 probes.

This dRT-PCR method has several advantages over conventional brute-force RT-PCR using degenerate primers. First and foremost, only those RT-PCR products that display differential expression between cell types or tissues are selected for sequencing. This greatly reduces the number of PCR products that must be sequenced for any set of degenerate primers to identify interesting genes. Second, the intensity of the hybridization signals exhibited by a given clone annealed with multiple probes, as well as compared to other clones annealed with the same probes, provides a unique “fingerprint” for that clone. In our experience, clones displaying similar fingerprints usually contain the same insert sequence. Thus, by selecting for sequencing only those clones which exhibit different hybridization fingerprints, the characterization of redundant inserts is reduced. Third, although sequencing of inserts from a PCR experiment may no longer be rate-limiting due to automation, the analysis of the expression pattern of each of the PCR products still represents a major bottleneck. dRT-PCR reduces the number of genes whose expression patterns have to be characterized by identifying differentially-expressed sequences at an early stage in the procedure. These three factors combined allow a relatively rapid assessment of whether a set of degenerate primers will identify gene family members differentially-expressed among a set of tissues or cell types. This in turn permits rapid screening of multiple pairs of degenerate primers, either within a single gene family or among several different families. Similar approaches related to dRT-PCR have been described previously by others (Boehm, 1993; Lai and Lemke, 1991; Wilkie and Simon, 1991).

An unexpected problem is that the in vivo expression pattern of several differentially-expressed dRT-PCR products was inconsistent with expectations based on their differential distribution in the original screen (T. Saito, L. Sommer and D.Anderson, unpublished observations). In some cases, a gene was not detectably expressed in the expected tissue but rather was highly expressed elsewhere. Similar situations were encountered in a screen of receptor tyrosine phosphatases (L. Sommer and D. Anderson, unpublished data).

The reason(s) for this discrepancy are not clear. It may reflect aberrant expression of genes in some of the cell lines used as sources of cDNA. Alternatively, in cases where dissected tissues were used it could reflect the efficient amplification of sequences present in minor or contaminating cell types. These are biological problems rather than problems with the dRT-PCR method per se, but they illustrate the importance of judiciously selecting multiple tissues or cell lines for the initial dRT-PCR screen. Another potential explanation is that some genes may be preferentially amplified by a specific primer set, which would be a problem intrinsic to the PCR method rather than to dRT-PCR. A final possibility, specific to dRT-PCR, derives from the fact that hybridization signal intensity in the clone blots actually reflects the relative abundance of that sequence within the gene family of interest, rather than in the cDNA population as a whole. In the limiting case where a given cell type expresses only a single family member, that sequence would represent 100% of the hybridization probe (and therefore yield an intense hybridization signal from that cell type) even if it is actually expressed at very low levels in that cell or tissue. Such artifacts can be minimized by performing RNase protection experiments for individual clones on different tissues. In any case, the pattern of specificity exhibited by the dRT-PCR procedure should be considered tentative until confirmed by in situ hybridization data. This in turn raises another problem, in that the short (typically <250 bp) PCR products provide insufficiently sensitive probes for the nonradioactive in situ hybridization procedure, necessitating additional time and effort to isolate longer cDNAs. The value of this method would be improved if sensitive and reliable non-radioactive in situ hybridization procedures could be performed directly with the PCR products. Nevertheless, the prescreening of clones in dRT-PCR still reduces the number of genes that have to be characterized by these more labor-intensive procedures. In summary, our data support the idea that transcription factors provide useful and specific markers for subtypes of neurons in the vertebrate nervous system. We have developed and applied a method that combines degenerate PCR amplification with differential hybridization to allow rapid screening of gene families for members which are differentially expressed among populations of neural cell types. This screen has yielded a novel paired homeodomain protein, DRG11, that represents the first sensory neuron-specific transcription factor identified in mammals.

cDNA library construction and screening Poly(A+) RNA was purified from 1 μg of total RNA from E13.5 rat dorsal root ganglion (DRG) by using oligo(dT) magnetic beads (Dynal), and converted to cDNA using the Superscript choice system (Gibco BRL). The cDNA was ligated to a pre-annealed mixture of oligonucleotides ACTGAAGCCAAGGTAGGATCCG and (phosphorylated) CGGATCCTACCTTGGCTTCAGTAG. The ligated cDNA was purified using the Spinbind PCR purification system (FMC) and amplified by 3 rounds of PCR (16 cycles, 12 cycles and 9 cycles) using a phosphorylated oligonucleotide (CTACTGAAGCCAAGGTAGGATCCG) der the condition of 95° C. for 1.5 min, 64° C. for 2 min, and 72° C. for 7 min. After each round of PCR, a small aliquot of the amplified DNA (one tenth of the total reaction mixture) was used for the next round of PCR. ³²P-dCTP was added in the third round of PCR to calculate the amount of amplified DNA. ˜1.3 μg of DNA was obtained after the third round of PCR. cDNA fragments longer than 500 bp were separated on a size-fractionation column (Gibco BRL) and cloned into the lambda ZapII vector (Stratagene).

The library was screened using a DNA probe containing the homeodomain of the DRG11 RT-PCR product. Two cDNA clones containing 2.4 kb and 1.0 kb inserts were obtained. Nucleotide sequences were determined on both strands using Sequenase (USB). The sequence of DRG11 has been submitted to GenBank under accession number U29174.

The deduced amino acid sequence of the longest cDNA (2.4 kb) obtained is presented in FIG. 3. The clone encodes a novel 28.6 Kd protein in the PHD family. As the 280 bp sequence upstream of the most N-terminal methionine contains an in-frame translational termination codon, this methionine has been tentatively assigned as the initiation codon. Attempts to determine the size of native DRG11 mRNA by Northern blotting of either total or polyA(+) RNA from rat embryos were unsuccessful, most likely due to the low abundance of the transcript. Moreover, the hamster anti-rat DRG11 monoclonal antibodies did not work well in Western blotting experiments; therefore the size of the native DRG11 protein has not been established. Thus, it cannot be ruled out that this protein is larger than that predicted by the deduced amino acid sequence from the cDNA clone.

DRG11 is closely related in sequence to Pax3 as well as to several other paired homeodomain proteins (FIG. 4). Unlike the Pax genes, however, DRG11 lacks a paired domain (FIG. 4). Interestingly, DRG11 has a Gln residue instead of a Ser at position 9 of the recognition helix (residue 56 in FIG. 4); a substitution of Gln for Ser at this position also occurs in several other family members that lack a paired domain (e.g., Phox2 and Cart1 in FIG. 4).

In Situ hybridization Non-radioactive in situ hybridization was performed as described previously (Birren et al., 1993). Following probes were used: SCG10 (Stein et al., 1988); trk A, from pDM97 (the gift of L. Parada); trkb, from pFRK16; trkc, from pRtrkc8 (the gifts of G. Yancopoulos). Islet-1 and PAX3 clones, which carry 0.9 and 1.2 kb of cDNA respectively, were obtained by RT-PCR.

The earliest stage at which DRG11 mRNA could be detected was at E12.5, 1-2 days following the initial condensation of neural crest cells to form dorsal root ganglia. Expression at this age was restricted to the nervous system, and within the trunk region was restricted to the dorsal root ganglia; no expression was detected in the neural tube (data not shown). By contrast, other transcription factors expressed in sensory neurons at this stage, such as Isl-1 and Pax3 are also expressed in the neural tube.

By E15.5, DRG11 expression was strong in trunk DRG (data not shown) and was also detected in the dorsal spinal cord. Positive cells were located both laterally near the dorsal root entry zone, and more medially near the ventricular zone. Between these two zones, there was a region containing only scattered DRG11-positive cells, although this region does contain neurons as shown by SCG10 hybridization. This pattern suggests that DRG11 is expressed by cells which display the known migration pattern of newly-born dorsal horn neurons (Langman and Haden, 1970): these cells are initially generated in the ventricular zone, then migrate laterally through an intermediate region to take up their final position at the dorso-lateral margins of the spinal cord, where they create a second region of high DRG11 expression. The DRG11 expression pattern is distinct from that of a related PHD protein, Pax3 (Goulding et al., 1991), which is also expressed in the dorsal spinal cord at this stage but only in the ventricular zone.

Although DRG11 was detected in sensory but not sympathetic ganglia (not shown) at E15.5, this apparent specificity could simply reflect the timing of differentiation in the two groups of neurons; sympathetic development is known to lag behind sensory development by several days. For example, trkA mRNA is initially expressed in sensory but not sympathetic neurons at E12.5 (Martin-Zanca et al., 1990), but is later detected in sympathetic ganglia beginning on E16.5-E17.5 (Birren et al., 1993). To determine whether this is also true for DRG11, the expression of DRG11 was examined in sections through the anterior trunk region of E17.5 embryos. These sections contain large sympathetic ganglia expressing SCG10, trkA and Isl-1, but not DRG11 (data not shown). This specificity of DRG11 expression in the PNS is also maintained at postnatal day 3 (not shown). At E17.5, DRG11 expression in the spinal cord appeared increased in the dorsal horns relative to E15.5. Interestingly, this region of the spinal cord receives synaptic input from the DRG (Kandel et al., 1991). Examination at high magnification of sections hybridized with the DRG11 cRNA probe revealed that the DRG11⁺ cells have a process-bearing morphology, suggesting that they are neurons (not shown).

These data suggest that DRG11 is expressed both by sensory neurons in the DRG and by a subset of their synaptic target neurons in the dorsal spinal cord. At no time, however, was DRG11 expression detected in the ventral spinal cord which also receives sensory innervation. No DRG11 expression was detected outside of the nervous system at any of the stages examined.

The expression of DRG11 in sensory ganglia as well as in a subset of their CNS targets raised the question of whether DRG11 is expressed only by those sensory neurons that project to the dorsal spinal cord. These neurons include the nociceptive, NGF-dependent subset (Ruit et al., 1992) which expresses trkA. These neurons can be distinguished from other sensory neurons which express trkC, are NT-3-dependent and project to the ventral spinal cord, many of which are proprioceptive (for review, see (Snider, 1994)). The expression of DRG11 in E13.5 trigeminal sensory ganglia as well as in trunk DRG appeared broader than that of trks A, B or C in nearby sections, however (data not shown). Rather, the extent of of DRG11 expression was similar to that of SCG10 (Stein et al., 1988) or Isl-1, two markers which label all neurons in these sensory ganglia. In addition, DRG11 expression was detected in both small and large DRG neurons at E17.5; in contrast the larger neurons expressed trksB and C but not A (data not shown). This suggests that, at least at this stage, DRG11 expression includes, but is not restricted to, the NGF-responsive subset of sensory neurons. That such neurons do express DRG11, however, is supported by double-label immunocytochemical labeling experiments (see below).

Antibody production DRG11 protein expressed in bacteria was gel-purified and used to immunize Armenian hamsters. Hamster spleen cells were fused with P3X63Ag8u.1 mouse myeloma cells. Supernatants were first screened on dot blots of recombinant DRG11 protein, and positives from this screen were rescreened by immunofluorescence staining of CHO cells transfected with a mammalian DRG11 expression construct.

To determine whether DRG11 expression within ganglia is restricted to neurons or is common to neurons and nonneuronal (glial) cells, its expression was examined in dissociated postnatal DRG cultures using the monoclonal antibody. Staining of perinatal rat dissociated DRG cultures revealed nuclear immunoreactivity in many sensory neurons, but not in glia. Approximately 60% of the neurons were labeled by the antibody under these conditions. Whether the apparent lack of DRG11 expression in other sensory neurons reflects distinct sensory lineages, or rather environmental modulation in culture, is currently being investigated. As an additional control for the specificity of the antibody, similar dissociated cultures of superior cervical sympathetic ganglia (SCG) were stained. No labeling of any cells was detected. Thus, as predicted by its initial selection in the dRT-PCR screen based on expression in DRG but not in MAH or NCM-1 cell cDNA, DRG11 is expressed in many sensory neurons but not in sympathetic neurons or glial cells.

The availability of a monoclonal antibody to DRG11 allowed a preliminary assessment of whether this transcription factor is indeed expressed by the trkA-expressing subset of DRG sensory neurons, as suggested by the in situ hybridization data. To address this question, dissociated cultures of E16.5 DRG were double-labeled with anti-DRG11 and a specific polyclonal antiserum to trkA (Clary et al., 1994). Many trkA⁺ neurons co-expressed DRG11 in their nuclei (data not shown). Conversely, it appeared that the majority of DRG11⁺ cells also expressed trkA, although a few DRG11⁺ trkA⁻ cells could be observed. Finally, some neurons in the cultures expressed neither marker. These data confirm that DRG11 is expressed by NGF-responsive DRG sensory neurons, many of which are nociceptive neurons that project to the dorsal horn of the spinal cord (Snider, 1994) where DRG11 is also expressed. However, DRG11 expression is also detected in some sensory neurons that do not express trkA, consistent with the in situ hybridization data.

The timing of DRG11 expression, 1-2 days after neurons are first detected in the DRG by expression of pan-neuronal markers such as SCG10, suggests that this putative transcriptional regulator is unlikey to be required for initial neuronal differentiation. Rather, this protein is likely to regulate later-developing aspects of sensory neuron phenotype or function. In this respect, the homology of DRG11 to another PHD protein, Phox2, is of interest. Phox2 is expressed specifically in developing autonomic ganglia but not in trunk sensory ganglia (Valarché et al., 1993), a pattern which is strikingly complementary to that of DRG11. Correlative data from in vivo and in vitro experiments, as well as DNA-binding data, suggest that Phox2 may be involved in the expression of neurotransmitter synthesizing enzymes such as DBH (Tissier-Seta et al., 1993). By analogy, DRG11 could play a role in specifying some aspect of the complex neurotransmitter phenotype of sensory neurons. Alternatively, if as suggested below, DRG11 function is important in appropriate synapse formation, its downstream targets could include cell surface proteins important in establishing or maintaining proper connectivity. In this respect it is of interest that Phox2 has also been shown to regulate the promoter of Ncam, a cell surface adhesion molecule (Tissier-Seta et al., 1993; Valarché et al., 1993).

An intruiging feature of DRG11 expression is that it is detected both in sensory neurons and in a subset of their target neurons in the spinal cord, specifically those in the dorsal horn. A subset of these central neurons receive input from the NGF-dependent population of sensory neurons, which (as shown by double-labeling with antibodies to trkA) also expresses DRG11. This suggests that DRG11 might function in regulating some aspect of synapse formation between sensory neurons and their central targets. However, DRG11 expression within sensory ganglia was more extensive than that of trkA; for example it was detected in large neurons which typically project to the ventral spinal cord (Snider, 1994). This suggests that DRG11 expression is not restricted to NGF-dependent sensory neurons. This indicates that DRG11 expression cannot be sufficient to specify the central connectivity of such neurons. However it could be necessary for some aspect of this process. This notion is strengthened by the fact that DRG11 first appears in the dorsal spinal cord at E15.5, about the time at which the first sensory afferents are growing into this region of the CNS. Studies in the chick indicate that some aspects of dorsal horn development depend upon sensory axon ingrowth (Sharma et al., 1994).

### BIBLIOGRAPHY

Begley et al., Proc. Natl. Acad. Sci. USA 89: 38-42.

Birren et al., (1990 Neuron 4: 189-201.

Birren et al. (1993) Development 119: 597-610.

Boehm, T. (1993). Oncogene 8:1385-1390.

Campbell, et al. (1994) Development 120: 2957-2966.

Campbell, et al. (1992) Genes & Dev. 6: 367-379.

Chomczynski, et al. (1987) Anal. Biochem. 162: 156-159.

Clary, et al., (1994) Mol. Biol. Cell 5: 549-563.

Ericson, et al., (1992) Science 256: 1555-1560.

Fann, et al., (1994) Proc. Natl. Acad. Sci. USA 91: 43-47.

Frigerio, et al., (1986) Cell 47: 735-746.

Gerrero, et al., (1993) Proc. Natl. Acad. Sci. USA 90: 10841-10845.

Göbel, et al., (1992) Cell Growth & Diff 3:143-148.

Goulding, et al., (1991) EMBO J 10:1135-1147.

Grueneberg, et al., (1992) Science 257: 1089-1095.

Ito, et al., (1993) Devel. Biol. 157: 517-525.

Johnson, et al., (1990). Nature 346:858-861.

Kandel, et al., (1991). Principles of Neural Science. Elsevier, New York.

Lai, C. and Lemke, G. (1991). Neuron 6:691-704.

Lai, et al., (1991) Genes & Dev. 5: 416-427.

Langman, et al., (1970) J. Comp. Neurol. 138: 419-431.

Lee, et al., (1995) Science 268: 836-844.

Libert, et al., (1989) Science 244: 569-572.

Lo, et al., (1994) Persp. Dev. Neuro. 2: 191-201.

Lo, et al., (1990) Devel. Biol. 145: 139-153.

Martin-Zanca, et al., (1990) Genes & Devel. 4: 683-694.

Matsumoto, S. G. (1994) Dev. Brain Res. 83: 17-27.

Miller, et al., (1992) Nature 355: 841-845.

Murphy, et al., (1991) Proc. Natl. Acad. Sci. USA 88: 3498-3501.

Nye, et al., (1992) Genes & Dev. 6: 975-990.

Opstelten, et al., (1991) Mech. Dev. 34: 29-41.

Rohrer, et al., (1987) J. Neurosci 7: 3739-3748.

Ruit, et al., (1992) Neuron 8: 573-587.

Sambrook, et al., (1989. Molecular cloning: A laboratory manual. Cold Spring Harbor Laboratory Press.

Schneitz, et al., (1993) Genes Dev. 7: 114-129.

Sharma, et al., (1994) Development 120: 1315-1323.

Snider, W. D. (1994) Cell 77: 627-638.

Stein, et al., (1988) Neuron 1: 463-476.

Tissier-Seta, et al., (1993) C.R. Acad. Sci. Paris 316: 1306-1315.

Tsuchida, et al., (1994) Cell 79: 957-970.

Valarché, et al., (1993) Development 119: 881-896.

Verdi, et al., (1994) Neuron 13: in press.

Walther, et al., (1991) Development 113: 1435-1449.

Wilkie, et al., (1991) METHODS: A Companion to Meth. Enzymol. 2: 32-41.

Xiong, et al., (1994) Genes Dev. 8: 981-994.

Zhao, et al., (1993) Proc. Natl. Acad. Sci. U.S.A. 90: 8633-8637.

We claim:

1. A recombinant nucleic acid encoding a DRG11 protein.

2. A recombinant nucleic acid according to claim 1 encoding the amino acid sequence depicted in FIG. 2.

3. A recombinant nucleic acid according to claim 1 which will hybridize to the nucleic acid depicted in FIG. 2.

4. A recombinant nucleic acid according to claim 1 comprising the nucleic acid depicted in FIG. 2.

5. An expression vector comprising transcriptional and translational regulatory DNA operably linked to DNA encoding a DRG11 protein.

6. A host cell transformed with an expression vector according to claim 5.

7. A method of producing a DRG11 protein comprising:
a) culturing a host cell transformed with an expressing vector comprising a nucleic acid encoding a DRG11 protein; and
b) expressing said nucleic acid to produce a DRG11 protein.

8. A recombinant DRG11 protein.

9. A recombinant DRG11 protein according to claim 8 encoded by a nucleic acid which hybridizes to the nucleic acid sequence shown in FIG. 2.

10. A recombinant DRG11 protein according to claim 8 which is at least about 80% homologous to the amino acid sequence shown in FIG. 3.

11. A recombinant DRG11 protein according to claim 8 which has the amino acid sequence shown in FIG. 3.

12. An antibody capable of specifically binding to a DRG11 protein.

13. A method for detecting a DRG11 protein in a target sample comprising contacting an antibody according to claim 12 with said target sample and assaying for the presence of binding between said polypeptide and DRG11, if present, in said target sample.

14. A method for determining the differential expression of a gene in different cell types or tissues comprising:
a) synthesizing libraries of nucleic acids from a plurality of different cell types or tissues using a set of primers;
b) subcloning a portion of the library obtained from a first of said different cell types or tissue to form a subclone library;
c) separately contacting members of said subclone library with probes each of which comprise one of said libraries wherein said nucleic acids in said libraries are labeled and wherein said contacting is under conditions which permit the hybridization of said labeled nucleic acids to complementary nucleic acids, if present, in said subclone library; and
d) determining whether hybridization has occurred for each of said probes for members of said subclone library as an indication of the differential expression of a gene expressed by said first cell type or tissue.

15. The method of claim 14 wherein said synthesizing comprises PCR amplification.

16. The method of claim 14 wherein each of the primers used in said synthesizing covers a conserved region of a family of genes.

17. The method of claim 16 wherein said conserved region is a homeodomain.
